(19) United States
(12) Patent Application Publication
(10) Pub. No.: US 2018/0230140 A1
(43) Pub. Date: Aug. 16, 2018

(54) MULTICOMPONENT CRYSTALS OF DASATINIB WITH MENTHOL OR VANILLIN

(71) Applicant: BASF SE, Ludwigshafen (DE)

(72) Inventors: Martin VIERTELHAUS, Mannheim (DE); Tiziana CHIODO, Mannheim (DE); Beate SALVADOR, Ellerstadt (DE); Marcus VOSSEN, Limburgerhof (DE); Andreas HAFNER, Gelterkinden (CH); Tobias HINTERMANN, Therwil (CH); Martin SZELAGIEWICZ, Basel (CH); Fritz BLATTER, Reinach (CH)

(73) Assignee: BASF SE, Ludwigshafen (DE)

(21) Appl. No.: 15/320,608

(22) PCT Filed: Jun. 24, 2015
(86) PCT No.: PCT/EP2015/064201

§ 371 Date: Dec. 20, 2016

(30) Foreign Application Priority Data
Jun. 30, 2014 (EP) 14174917.6
Mar. 19, 2015 (EP) 15159835.6

(51) Int. Cl. C07D 417/14; C07C 39/06; C07C 47/58; A61P 35/02

(52) U.S. Cl. CPC C07D 417/14; C07C 39/06; C07B 2200/13; A61P 35/02; C07C 47/58

(57) ABSTRACT

The present invention primarily relates to multicomponent crystals comprising a compound of formula 1 and a second compound selected from the group consisting of menthol and vanillin. The invention is further related to pharmaceutical compositions comprising such multicomponent crystals. Furthermore, the invention relates to processes for preparing said multicomponent crystals. The invention also relates to several aspects of using said multicomponent crystals or pharmaceutical compositions to treat a disease.

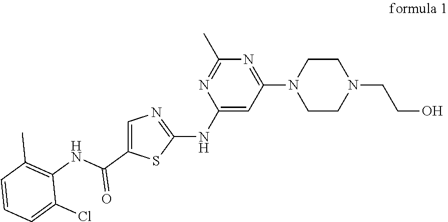 [SMILES: Cc1nc(Nc2ncc(C(=O)Nc3c(C)cccc3Cl)s2)cc(N2CCN(CCO)CC2)n1]

Dasatinib which is also known as BMS-354825 was disclosed in WO Patent Publication No. 00/62778 and in U.S. Pat. No. 6,596,746. Dasatinib, chemically N-(2-chloro-6-methylphenyl)-2-[[6-[4-(2-hydroxyethyl)-1-piperazinyl]-2-methyl-4-pyrimidinyl]amino]-5-thiazolecarboxamide, is represented by the following structure:

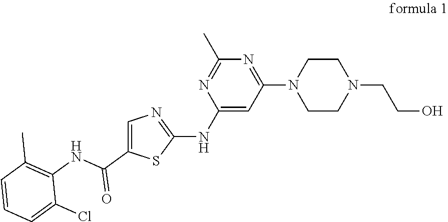 [SMILES: Cc1nc(Nc2ncc(C(=O)Nc3c(C)cccc3Cl)s2)cc(N2CCN(CCO)CC2)n1]

Dasatinib is a drug produced by Bristol-Myers Squibb and sold under the trade name Sprycel® (which contains Dasatinib monohydrate as the active ingredient). Dasatinib is an oral dual BCR/ABL and Src family tyrosine kinase inhibitor approved for use in patients with chronic myelogenous leukemia (CML) after imatinib treatment and Philadelphia chromosome-positive acute lymphoblastic leukemia (Ph+ ALL).

The present invention primarily relates to a process for obtaining multicomponent crystals comprising a compound of formula 1 (cf. above) and a second compound selected from the group consisting of menthol and vanillin, and to the multicomponent crystals thus obtained or obtainable.

The invention is further related to pharmaceutical compositions comprising said multicomponent crystals. The invention also relates to several aspects of using said multicomponent crystals or pharmaceutical compositions to treat a disease. Further details as well as further aspects of the present invention will be described herein below.

A compound like Dasatinib may give rise to a variety of crystalline forms having distinct crystal structures and physical characteristics like melting point, X-ray diffraction pattern, infrared spectrum, Raman spectrum, and solid state NMR spectrum. One crystalline form may give rise to thermal behavior different from that of another crystalline form. Thermal behavior can be measured in the laboratory by such techniques as capillary melting point, thermogravimetric analysis (TGA), and differential scanning calorimetry (DSC) as well as content of solvent in the crystalline form, which have been used to distinguish polymorphic forms.

Dasatinib is known to exist in close to 60 solid-state forms: a monohydrate, four anhydrous and unsolvated forms which are described in U.S. Pat. No. 7,491,725B2, US2006/0004067A1, U.S. Pat. No. 7,973,045B2, and WO2010/067374, and therein referred to as forms N-6, T1H1-7, B, and I. Further forms (such as 52 solvates) are known from WO2007/035874, US2006/0004067A, WO2009/053854A2, U.S. Pat. No. 8,067,423B, WO2010/062715, and CN102030745. In particular, patent application WO 2010/062715 includes the solvents isosorbide dimethyl ether, N,N′-dimethylethylene urea and N,N′-dimethyl-N,N′-propylene urea. Isosorbide dimethyl ether is used in cosmetic and pharmaceutical formulations.

The discovery of new forms of a pharmaceutically useful compound offers an opportunity to improve the performance profile of a pharmaceutical product. It widens the reservoir of materials a formulation scientist has available for designing a new dosage form of a drug with improved characteristics.

Co-crystals comprising Dasatinib and selected co-crystal formers have been described in WO2013/186726.

Due to the strong tendency of Dasatinib to form solvates an economic process for preparation of a co-crystal can hardly be achieved from solution as the solvate formation is in competition to the co-crystal formation. Methanol is the only solvent that dissolves Dasatinib in a reasonable concentration. However, methanol is an ICH class 2 solvent and thus is restricted for use in pharmaceutical products and has to be specially controlled (see International Conference on Harmonisation of Technical Requirements for Registration of Pharmaceuticals for Human Use (ICH), Impurities: Guideline for Residual Solvents Q3C(R5) of 4 Feb. 2011). Furthermore, methanol is the solvent that forms the solvate with the lowest stability. This means that the methanolate is the solvate that can be desolvated easiest.

Therefore, there is a need for a preparation process for co-crystals comprising Dasatinib that avoids the above disadvantages, and allows for a preparation without the need of using solvents, or using an ICH class 3 solvent only. ICH class 3 solvents include Acetic acid, Heptane, Acetone, Isobutyl acetate, Anisole, Isopropyl acetate, 1-Butanol, Methyl acetate, 2-Butanol, 3-Methyl-1-butanol, Butyl acetate, Methylethyl ketone, tert-Butylmethyl ether (MTBE), Methyl-isobutyl ketone, Dimethyl sulfoxide, 2-Methyl-1-propanol, Ethanol Pentane, Ethyl acetate, 1-Pentanol, Ethyl ether, 1-Propanol, Ethyl formate, 2-Propanol, Formic acid, Propyl acetate. According to a preferred objective in connection with the present invention, the obtained crystalline forms are essentially free of residual solvent.

Surprisingly, a new procedure was found to produce co-crystals of Dasatinib with menthol or vanillin using only menthol or vanillin and, optionally, an ICH class 3 solvent for removal of excess co-crystal former, without the need for evaporation of the solvent at elevated temperatures, resulting in a very low residual solvent content without significant loss of the co-crystal former. This procedure can be used to produce co-crystals of Dasatinib with menthol or vanillin in high purity and at larger scale starting with any form of Dasatinib.

### SUMMARY OF THE INVENTION

The invention provides a new process for obtaining multicomponent crystals comprising a compound of formula 1 (INN: Dasatinib)

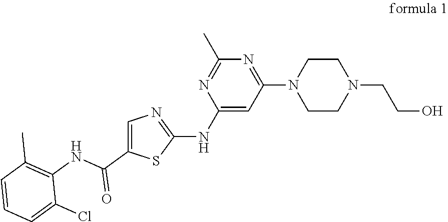 [SMILES: Cc1nc(Nc2ncc(C(=O)Nc3c(C)cccc3Cl)s2)cc(N2CCN(CCO)CC2)n1]

and

a second compound selected from the group consisting of menthol and vanillin, the process comprising the steps of:

a) providing a compound of formula 1 (INN: Dasatinib)

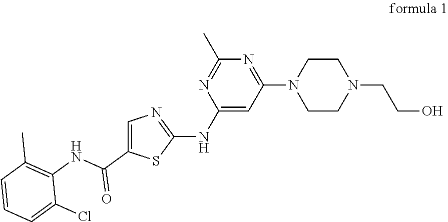 [SMILES: Cc1nc(Nc2ncc(C(=O)Nc3c(C)cccc3Cl)s2)cc(N2CCN(CCO)CC2)n1]

b) adding menthol or vanillin to the compound of step a) in an amount that is at least stoichiometric, preferably substantially greater than the amount in the obtained co-crystals,

and new multicomponent crystals thus obtained.

Novel pharmaceutical compositions containing these multicomponent crystals as well as aspects of using said multicomponent crystals or compositions to treat a disease are also described herein.

### DETAILED DESCRIPTION OF THE INVENTION

The present invention is directed to a process for obtaining multicomponent crystals comprising a compound of formula 1 (INN: Dasatinib)

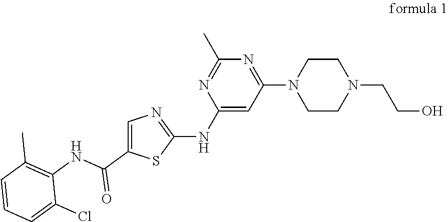 [SMILES: Cc1nc(Nc2ncc(C(=O)Nc3c(C)cccc3Cl)s2)cc(N2CCN(CCO)CC2)n1]

and

a second compound selected from the group consisting of menthol and vanillin, the process comprising the steps of:

a) providing a compound of formula 1 (INN: Dasatinib)

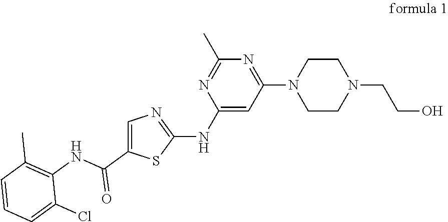 [SMILES: Cc1nc(Nc2ncc(C(=O)Nc3c(C)cccc3Cl)s2)cc(N2CCN(CCO)CC2)n1]

b) adding menthol or vanillin (hereinafter also referred to as co-crystal former) to the compound of step a) in an amount that is at least stoichiometric, preferably substantially greater than the amount in the obtained co-crystals.

Substantially greater in the context of the present invention means that when the pure co-crystal former is used as the solvent the amount needs to be sufficient in order to achieve a suspension that can be stirred. If the process is carried out with an additional solvent then substantially greater means that concentration of co-crystal former in the suspension is above the critical activity for co-crystal formation. Thus, in each of these cases, the co-crystal former is added in excess, i.e. the amount of co-crystal former added in step b) advantageously is substantially greater than the amount in the obtained co-crystals. Typically at least 2 molar parts of co-crystal former are added on one molar part of compound of formula 1; more preferably, about 1 part by weight of co-crystal former or more, for example about 1 to 20 parts by weight or even about 1 to 10 parts by weight of co-crystal former, is added in step b) on one part by weight of the compound of formula 1.

Preferably, the co-crystal former menthol or vanillin is used as the solvent, and the temperature of operation corresponds at least to the melting temperature of the co-crystal former. Optionally, a suitable solvent as noted above is added that allows working at lower temperatures for suspension equilibration and filtration. The process of the invention thus advantageously comprises a step (c) of heating the mixture obtained in step (b), e.g. to a temperature from the range 40 to 150° C., typically under agitating such as stirring, and a step (d) of cooling, e.g. to a temperature from the range −10° C. to less than 30° C., for example room temperature, and a step (e) of isolating the crystalline material obtained, e.g. by decantation, filtration or centrifugation, with optional washing of the material with an ICH class 3 solvent, especially with the solvent noted above for step (b) or with a solution of the co-crystal former in the solvent.

Heating of the mixture is advantageously carried out under exclusion of oxygen atmosphere, e.g. by purging with nitrogen.

Typically, the mixture obtained in step (b) is thus heated above 30° C., especially to 40-150° C., in case that the co-crystal former and solvent is menthol; or above 80° C., especially to 82-150° C., in case that the co-crystal former and solvent is vanillin; or above 30° C., especially to 35-110° C., in case that the co-crystal former is menthol or vanillin and the additional solvent is used.

In case that any additional solvent is added, such solvent is selected from C2 to C5 alcohols of which are particularly preferred ethanol, and propanol; C3 to C6 ketones of which are particularly preferred acetone, methyl ethyl ketone and methyl isobutyl ketone; C2 to C6 esters of which are particularly preferred methyl acetate, ethyl acetate, propyl acetate, butyl acetate, ethyl formate; ethers, typically C2-C10 ethers, of which are particularly preferred ethyl ether, and most preferred methyl tert-butyl ether; alkanes, typically C2-C12 alkanes, of which are particularly preferred pentane and heptane.

Preferably, co-crystal formation is achieved in a suspension or concentrated solution of the co-crystal former menthol or vanillin in a suitable solvent as noted above. Also preferred is co-crystal formation in a suspension or solution of Dasatinib in menthol as the co-crystal former and only solvent.

In a preferred embodiment, the process described herein further comprises the steps of:

c) heating the composition obtained in step b) to a temperature that exceeds the melting temperature of the co-crystal former;

d) stirring the suspension obtained in step c) at a temperature that exceeds the melting temperature of the co-crystal former;

e) filtrating the suspension obtained in step d);

f) cooling the solid obtained in step e) to near ambient temperature;

g) optionally washing the solid obtained in step f) with a solvent or with a solution of the co-crystal former in a solvent, typically to remove unreacted co-crystal former;

h) optionally filtrating the composition obtained in step g);

i) drying the obtained solid.

In another preferred embodiment, the process described herein further comprises the steps of:

c) heating the composition obtained in step b) to a temperature that exceeds the melting temperature of the co-crystal former;

d) stirring the suspension obtained in step c) at a temperature that exceeds the melting temperature of the co-crystal former;

e) cooling the suspension obtained in step d) to near ambient temperature;

f) adding a solvent to the composition of step e);

g) stirring the suspension obtained in step f) near ambient temperature;

h) filtrating the composition obtained in step g);

i) optionally washing the solid obtained in step h) with a solvent or with a solution of the co-crystal former in a solvent, typically to remove unreacted co-crystal former;

j) optionally filtrating the composition obtained in step i);

k) drying the obtained solid.

In a further preferred embodiment, the process described herein further comprises the steps of:

c) adding a solvent to the composition obtained in step b) and stirring the obtained mixture, preferably at a temperature around or below the boiling point of the added solvent;

d) optionally cooling the suspension obtained in c) to near ambient temperature;

e) stirring the suspension obtained in step d) near ambient temperature;

f) filtrating the suspension obtained in step c) or e);

g) optionally washing the solid obtained in step f) with a solvent or with a solution of the co-crystal former in a solvent;

h) optionally filtrating the composition obtained in step g);

i) drying the obtained solid.

In the process described herein any solvent used besides menthol and vanillin preferably is an ICH class 3 solvent, more preferably methyl tert-butyl ether (MTBE).

After heating and/or suspending the mixture of compound of formula 1 and the co-crystal former until crystallization sets in, seed crystals may be added, though such addition is not necessary in the process of the invention.

In the process described herein menthol preferably is (1R,2S,5R)-(−)-menthol or (1S,2R,5S)-(+)-menthol or DL-menthol or a stereoisomer of menthol or a mixture thereof.

The present invention is also directed to multicomponent crystals comprising a compound of formula 1 (INN: Dasatinib)

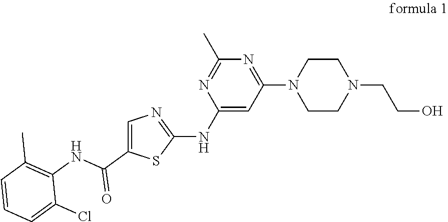 [SMILES: Cc1nc(Nc2ncc(C(=O)Nc3c(C)cccc3Cl)s2)cc(N2CCN(CCO)CC2)n1]

and

a second compound selected from the group consisting of menthol and vanillin obtained or obtainable by a process described herein.

Preferably, the multicomponent crystals are characterized in that the molar ratio of Dasatinib to the second compound is in the range of from 1.1:1 to 0.9:1, if vanillin is the second compound, preferably about 1:1. If menthol is the second compound, the ratio is from the range 2.2:1 to 1.8:1, and the ratio preferably is about 2:1.

In a preferred embodiment, the second compound is menthol and the multicomponent crystal has a PXRD pattern with at least one, preferably more or all characteristic peak(s) (expressed in °2θ±0.2° 2θ (CuKα radiation)) selected from the following peaks located at 5.8, 9.1, 10.4, 11.7, 11.9, 12.7, 14.9, 15.7, 16.5, 18.2, 21.1, 21.3, 21.8, 22.8, 23.9, 24.4.

In a preferred embodiment, the second compound is vanillin and the multicomponent crystal has a PXRD pattern with at least one, preferably more or all characteristic peak(s) (expressed in °2θ±0.2° 2θ (CuKα radiation)) selected from the following peaks located at 5.9, 8.9, 11.2, 11.8, 12.9, 15.4, 16.0, 17.7, 17.9, 18.6, 19.0, 19.8, 20.7, 22.4, 24.0, 24.6, 25.4, 26.3.

The present process allows for the preparation of co-crystals of the compound of formula 1 and menthol or vanillin (i.e. multicomponent crystals) in high purity, especially with regard to undesired constituents: Since the crystallization of the active agent Dasatinib constitutes the last step in its production, any remaining constituents normally find their way into the medicament finally applied. The present process allows for a limitation of such remaining constituents to ICH class 3 solvents. The present process further allows for the exclusion of pure crystalline Dasatinib, since this active agent is fully converted into the present co-crystal; in consequence, none of the unwanted properties of pure Dasatinib such as poor solubility are shown by the product of the present process, and no Dasatinib crystals are remaining which might act as seeds for the reconversion of the present co-crystal into its educts. For this goal, it may be advantageous to provide a certain excess amount of the co-crystal former (menthol or vanillin) in the product finally isolated in the process of the invention.

The invention thus further pertains to a composition essentially consisting of the multicomponent crystal according to the invention and the co-crystal former contained in said multicomponent crystal, or to a composition essentially consisting of the multicomponent crystal. The term “essentially consisting of” means that the present composition may contain only minor amounts of any further component, such as ICH class 3 solvents and water, besides the compound of formula 1 and menthol or vanillin; “minor amount” typically means an amount of less than 5%, for example less than 1%, and especially less than 0.5%, by weight of the total composition. The amount of excess co-crystal former is typically less than 50% by weight of the total composition, preferably 0.1 to 20% by weight, especially 0.1 to 10% by weight of the total composition.

The weight amount of any other impurity is typically below 10 ppm, especially below 1 ppm. The amount of residual ICH class 3 solvent is typically below 0.5% by weight, especially below 0.1% by weight. The composition essentially consisting of the multicomponent crystal according to the invention thus typically consists of 50 to 99.9%, especially 80 to 99.9%, by weight of co-crystal material consisting of the compound of formula 1 and the co-crystal former in the molar ratio forming the crystalline lattice of the present co-crystals, and 0.1 to 50%, especially 0.1 to 20% by weight of additional components. These 0.1 to 50% or especially 0.1 to 20% by weight of additional components generally consist of co-crystal former while a minor amount (up to 0.5%) may be residual ICH class 3 solvent and water, and the amount of compound of formula 1 in its prior crystalline form, or of any further impurity, generally is less than 0.1% by weight or zero.

The multicomponent crystals of the present invention are generally obtained as a fine powder with typical particle size distributions with the median size between 0.1 and 100 μm, preferably between 1 and 50 μm, preferably between 1 to 10 μm [crystal size as determined e.g. by the single-particle optical sensing (SPOS) method (AccuSizer 780/A, Particle Sizing Systems)]. This particle size range ensures a fast dissolution profile, while retaining the favorable handling properties in the formulation process.

The multicomponent crystals of the present invention may be used in pharmaceutical compositions in the same way as other forms of Dasatinib previously known. Additionally, the present multicomponent crystals may be employed as intermediates or starting materials to produce the pure active ingredient.

A further aspect of the present invention is a pharmaceutical composition comprising, as active ingredient, multicomponent crystals according to the present invention, preferably multicomponent crystals as described herein above as being preferred, and preferably further comprising one, two, three, or more pharmaceutically acceptable carriers, and/or diluents, and/or further ingredients, in particular one, two, three, or more pharmaceutical excipients.

The amount of the multicomponent crystals in the composition depends on the type of formulation and the desired dosage regimen during administration time periods. The amount in each oral formulation may be from 0.1 to 300 mg, preferably from 1.0 to 250 mg, in particular from 5.0 to 200 mg.

Oral formulations (as preferred pharmaceutical compositions according to the present invention) may be solid formulations such as capsules, tablets, pills and troches, or a liquid suspension formulation.

The multicomponent crystals according to the invention may be used directly in the form of powders, granules, suspensions, or they may be combined together with other pharmaceutically acceptable ingredients in admixing the components and optionally finely divide them, and then filling capsules, composed for example from hard or soft gelatin, compressing tablets, pills or troches, or suspend in suspensions. Coatings may be applied after compression to form pills.

Pharmaceutically acceptable ingredients are well known for the various types of formulation and may be for example binders such as natural or synthetic polymers, excipients, disintegrants, lubricants, surfactants, sweetening and other flavouring agents, coating materials, preservatives, dyes, thickeners, adjuvants, antimicrobial agents and carriers for the various formulation types.

Examples for binders are gum tragacanth, acacia, starch, gelatin, and biological degradable polymers such as homo- or co-polyesters of dicarboxylic acids, alkylene glycols, polyalkylene glycols and/or aliphatic hydroxyl carboxylic acids; homo- or co-polyamides of dicarboxylic acids, alkylene diamines, and/or aliphatic amino carboxylic acids; corresponding polyester-polyamide-co-polymers, polyanhydrides, polyorthoesters, polyphosphazene and polycarbonates. The biological degradable polymers may be linear, branched or crosslinked. Specific examples are poly-glycolic acid, poly-lactic acid, and poly-d,l-lactide/glycolide. Other examples for polymers are water-soluble polymers such as polyoxaalkylenes (polyoxaethylene, polyoxapropylene and mixed polymers thereof, poly-acrylamides and hydroxylalkylated polyacrylamides, poly-maleic acid and esters or -amides thereof, poly-acrylic acid and esters or -amides thereof, poly-vinylalcohol und esters or -ethers thereof, poly-vinylimidazole, poly-vinylpyrrolidon, und natural polymers like chitosan, carragenan or hyaluronic acid.

Examples for excipients are phosphates such as dicalcium phosphate.

Examples for disintegrants are croscarmellose sodium, crospovidone, low-substituted hydroxypropyl cellulose, sodium starch glycolate or alginic acid.

Surfactants may be anionic, cationic, amphoteric or neutral. Examples for surfactants are lecithin, phospholipids, octyl sulfate, decyl sulfate, dodecyl sulfate, tetradecyl sulfate, hexadecyl sulfate and octadecyl sulfate, Na oleate or Na caprate, 1-acylaminoethane-2-sulfonic acids, such as 1-octanoylaminoethane-2-sulfonic acid, 1-decanoylaminoethane-2-sulfonic acid, 1-dodecanoylaminoethane-2-sulfonic acid, 1-tetradecanoylaminoethane-2-sulfonic acid, 1-hexadecanoylaminoethane-2-sulfonic acid, and 1-octadecanoylaminoethane-2-sulfonic acid, and taurocholic acid and taurodeoxycholic acid, bile acids and their salts, such as cholic acid, deoxycholic acid and sodium glycocholates, sodium caprate or sodium laurate, sodium oleate, sodium lauryl sulphate, sodium cetyl sulphate, sulfated castor oil and sodium dioctylsulfosuccinate, cocamidopropylbetaine and laurylbetaine, fatty alcohols, cholesterols, glycerol mono- or -distearate, glycerol mono- or -dioleate and glycerol mono- or -dipalmitate, and poly-oxyethylene stearate.

Examples for sweetening agents are sucrose, fructose, lactose or aspartam.

Examples for flavouring agents are peppermint, oil of wintergreen or fruit flavours like cherry or orange flavour.

Examples for coating materials are gelatin, wax, shellac, sugar or biological degradable polymers.

Examples for preservatives are methyl or propylparabens, sorbic acid, chlorobutanol, phenol and thimerosal.

Examples for adjuvants are fragrances.

Examples for thickeners are synthetic polymers, fatty acids and fatty acid salts and esters and fatty alcohols.

Examples for solid carriers are talc, clay, microcrystalline cellulose, silica, alumina and the like.

The formulation according to the invention may also contain isotonic agents, such as sugars, buffers or sodium chloride.

The compositions of the present invention may also be formulated as effervescent tablet or powder, which can disintegrate in an aqueous environment to provide a drinking solution.

The most preferred route is oral administration. The dosages may be conveniently presented in a unit dosage form and prepared by any of the methods well-known in the art of pharmacy.

Capsule dosages, of course, will contain the solid composition within a capsule which may be made of gelatin or other conventional encapsulating material. Tablets and powders may be coated. Tablets and powders may be coated with an enteric coating. The enteric coated powder forms may have coatings comprising phthalic acid cellulose acetate, hydroxypropylmethyl-cellulose phthalate, polyvinyl alcohol phthalate, carboxymethylethylcellulose, a copolymer of styrene and maleic acid, a copolymer of methacrylic acid and methyl methacrylate, and like materials, and if desired, they may be employed with suitable plasticizers and/or extending agents. A coated tablet may have a coating on the surface of the tablet or may be a tablet comprising a powder or granules with an enteric-coating.

The multicomponent crystals of the present invention and the formulations or compositions containing the same, respectively, can also be administered in combination with other therapeutic agents being effective to treat a given condition and/or to provide a combination therapy.

The multicomponent crystals of the present invention and the pharmaceutical compositions according to the invention are useful for effective treatment of disorders in connection with need of inhibiting the BCR/ABL and Src family tyrosine kinases. The multicomponent crystals of the present invention and the respective pharmaceutical compositions are useful in the treatment of chronic myelogenous leukemia but also advanced prostate cancer.

The multicomponent crystals of the present invention and the pharmaceutical compositions according to the invention can also be used in a therapeutic method for producing an Abl tyrosine kinase inhibiting effect in a mammal comprising administering to a mammal in need of such therapy.

The multicomponent crystals of the present invention may be used as single component or as mixtures with other solid forms.

In view of the above, the present invention also relates to multicomponent crystals of the present invention and pharmaceutical compositions according to the invention for use as a medicament, preferably for use in the treatment of cancer, in particular of chronic myelogenous leukemia (CML) and/or Philadelphia chromosome-positive acute lymphoblastic leukemia (Ph+ ALL).

In the following, the present invention will be described more closely by way of selected examples illustrating the invention.

Wherever noted, in the following, room temperature depicts a temperature from the range 22-25° C., ambient temperature is defined as 25±10° C. and percentages are given by weight, if not indicated otherwise.

Abbreviations:

DMSO dimethyl sulfoxide

NMR nuclear magnetic resonance

TG-FTIR thermogravimetry coupled with Fourier-transformation infrared spectrometry

r.h. relative humidity (air, if not indicated otherwise)

TGA thermogravimetry

v/v volume by volume

PXRD powder X-ray diffraction

MTBE methyl tert-butyl ether

DSC differential scanning calorimetry

Instrumental:

Powder X-Ray Diffraction:

The measurements were carried out with a Panalytical X'Pert Pro diffractometer (manufacturer: Panalytical) using Cu Kα radiation in the Bragg-Brentano reflection geometry. Generally, the 2θ values are accurate within an error of ±0.1-0.2°. The relative peak intensities can vary considerably for different samples of the same crystalline form because of different preferred orientations of the crystals. The samples were prepared without any special treatment other than the application of slight pressure to get a flat surface. Generally, silicon single crystal sample holders of 0.1-1.0 mm depth were used. The tube voltage and current were 45 kV and 40 mA, respectively. Diffraction patterns were recorded in the range from 2θ=3°-35° with increments of 0.0167°. The samples were rotated during the measurement.

Thermogravimetry Coupled to Infrared Spectroscopy (TG-FTIR):

Thermogravimetry coupled with FT-infrared spectroscopy is a well known method that allows to monitor the mass loss of a given sample upon heating while identifiying the volatile substances by infrared spectroscopy. Therefore, TG-FTIR is a suitable method to identify solvates or hydrates.

TG-FTIR was performed on a Netzsch Thermo-Microbalance TG 209, which is coupled to a Bruker FT-IR Spectrometer Vector 22 or IFS 28. The measurements were carried out with aluminum crucibles with a micro pinhole under a nitrogen atmosphere and at a heating rate of 10° C./min over the range 25-250° C. or 25-350° C.

Differential Scanning Calorimetry (DSC):

Differential scanning calorimetry was carried out with a TA Instruments DSC Q2000 using hermetically sealed gold sample pans. The heating rate was 10° C. per minute and the samples were treated for three minutes under nitrogen before the sample pans were closed under nitrogen.

¹H-NMR:

The ¹H-NMR spectra were recorded on a Bruker DPX 300 spectrometer.

Solvent: Deuterated-DMSO (dimethyl sulfoxide-d₆)

Solvents: For all experiments, standard grade solvents are used.

### EXAMPLES

### Example 1

0.507 g of Dasatinib (monohydrate form) and 4.5 g (1R,2S,5R)-(−)-menthol are placed in a 15 mL glass vial. The gas phase is purged by dry nitrogen and the sample is heated to 120° C. in about one hour, stirred at about 120° C. for about 0.1 hour and cooled to 90° C. A slight dry nitrogen flow is used during both the heating and cooling phases. The solid material is separated by hot filtration at about 90° C. and cooled to room temperature on the filter. The solid material is resuspended on the filter fora short time using 10 mL of MTBE and filtered. The resuspension and filtration step is then repeated two more times and the solid material is subsequently air dried at room temperature for about 10 minutes.

Yield: about 0.5 g.

The PXRD pattern complies with the pattern shown in FIG. 1.

H-NMR spectroscopy indicates a molar ratio of dasatinib to menthol of about 2:1.

TG-FTIR shows a mass loss step between about 150° C. and 250° C. of about 13.8% (menthol) which confirms a 2:1 ratio Dasatinib:menthol (theor. 13.8%).

The onset of the first endothermal peak in DSC (about 39 J/g) is observed at about 166° C.

### Example 2

0.508 g of Dasatinib (monohydrate form) and 4.5 g (1R,2S,5R)-(−)-menthol are placed in a 40 mL glass vial. The gas phase is purged by dry nitrogen and the sample is heated to 80° C. The suspension is stirred at 80° C. for 3 hours and then cooled to room temperature. A slight dry nitrogen flow is used during both the heating and cooling phases. 20 mL of MTBE are added and the suspension is stirred for 0.5 hour at room temperature. The suspension is filtered and the solid material air dried. The solid material is resuspended on the filter for a short time using 10 mL of MTBE containing 25 mg/mL menthol, and filtered. The resuspension and filtration step is then repeated two more times and the solid material is subsequently air dried at room temperature for about 10 minutes.

Yield: about 0.5 g.

The PXRD pattern complies with the pattern shown in FIG. 1.

H-NMR spectroscopy indicates a molar ratio of Dasatinib to menthol of about 2:1.

TG-FTIR shows a mass loss step between about 140° C. and 250° C. of about 14.1% (menthol) which confirms a 2:1 ratio Dasatinib:menthol (theor. 13.8%).

The onset of the first endothermal peak in DSC (about 40 J/g) is observed at about 168° C.

### Example 3

5.198 g of dasatinib (monohydrate form) and 45.03 g of (1R,2S,5R)-(−)-menthol are placed in a 500 mL glass reactor. The gas phase is purged by dry nitrogen and the sample is heated to 80° C. The suspension is stirred at 80° C. for 5 hours and then cooled to room temperature. A slight dry nitrogen flow is used during both the heating and cooling phases. 200 mL of MTBE are added and the suspension is stirred for 0.5 hour at room temperature. The suspension is filtered and the solid material air dried. The solid material is resuspended on the filter for a short time using 100 mL of MTBE and filtered. The resuspension and filtration step is then repeated two more times and the solid material is subsequently air dried at room temperature for about 10 minutes.

Yield: about 5.6 g.

The PXRD pattern complies with the pattern shown in FIG. 1.

H-NMR spectroscopy indicates a molar ratio of Dasatinib to menthol of about 2:1.

TG-FTIR shows a mass loss step between about 140° C. and 210° C. of about 13.6% (menthol) which confirms a 2:1 ratio Dasatinib:menthol (theor. 13.8%).

The onset of the first endothermal peak in DSC (about 41 J/g) is observed at about 167° C.

Single crystals of Dasatinib-menthol co-crystal (2:1) are obtained. The stoichiometry of the co-crystal can be proven by the crystal structure.

### Example 4

0.249 g of Dasatinib (monohydrate form) and 0.253 g vanillin are placed in a 15 mL glass vial. The gas phase is purged by dry nitrogen and the sample is heated to 110° C. in about 0.5 hour. The suspension is stirred at 110° C. for about 0.5 hour and cooled to room temperature. A slight dry nitrogen flow is used during both the heating and cooling phases. 3.0 mL of MTBE are added and the suspension is stirred for 15 minutes at room temperature. The suspension is filtered and the solid material air dried. The solid material is resuspended on the filter for a short time using 3 mL of MTBE, filtered and air dried at room temperature for about 3 minutes.

Yield: about 0.18 g.

The PXRD pattern complies with the pattern shown in FIG. 2.

H-NMR spectroscopy indicates a molar ratio of Dasatinib to vanillin of about 1:1.

TG-FTIR shows a mass loss step between 25° C. and about 130° C. of about 0.6% (MTBE). The onset of the first very weak endothermal peak in DSC (about 1J/g) is about 67° C. and the onset of the second endothermal peak (about 48 J/g) is observed at about 155° C.

### Example 5

8.06 g of dasatinib (monohydrate form), 52.0 g of vanillin and 160 mL of MTBE are placed in a 350 mL glass reactor and heated to 55° C. The suspension is stirred at 55° C. for 3 days and then cooled to room temperature at a rate of about 20 K/hour. The suspension is then stirred at room temperature for 4.5 hours and filtered. The solid material is air dried, resuspended on the filter for a short time using 800 mL of MTBE containing 25 mg of vanillin/mL and filtered. The resuspension and filtration step is then repeated one more time and the solid material is subsequently air dried at room temperature for about 10 minutes.

Yield: 9.0 g.

The PXRD pattern complies with the pattern shown in FIG. 2.

H-NMR spectroscopy indicates a molar ratio of dasatinib to vanillin of about 1:1.

TG-FTIR shows a mass loss step between 25° C. and about 120° C. of less than 0.3%.

The onset of the first very weak endothermal peak in DSC (about 3 J/g) is about 78° C. and the onset of the second endothermal peak (about 44 J/g) is observed at about 154° C.

Single crystals of Dasatinib-vanillin co-crystal (1:1) are obtained. The stoichiometry of the co-crystal can be proven by the crystal structure.

### BRIEF DESCRIPTION OF FIGURES

FIG. 1: PXRD pattern of Dasatinib-menthol co-crystal 2:1 (CuKα radiation)

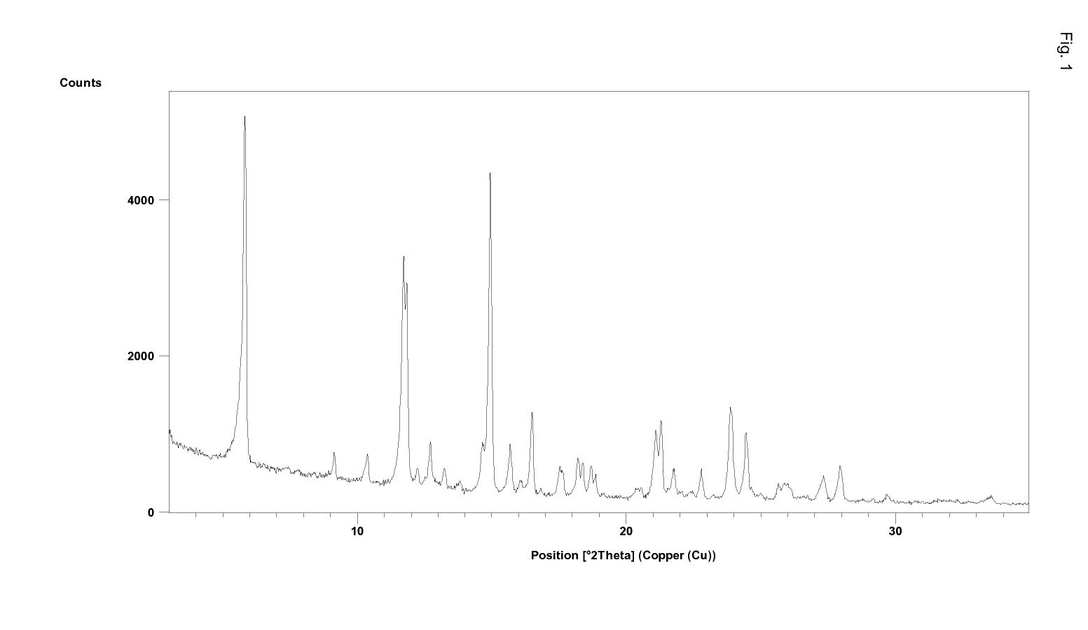

FIG. 2: PXRD pattern of Dasatinib-vanillin co-crystal 1:1 (CuKα radiation)

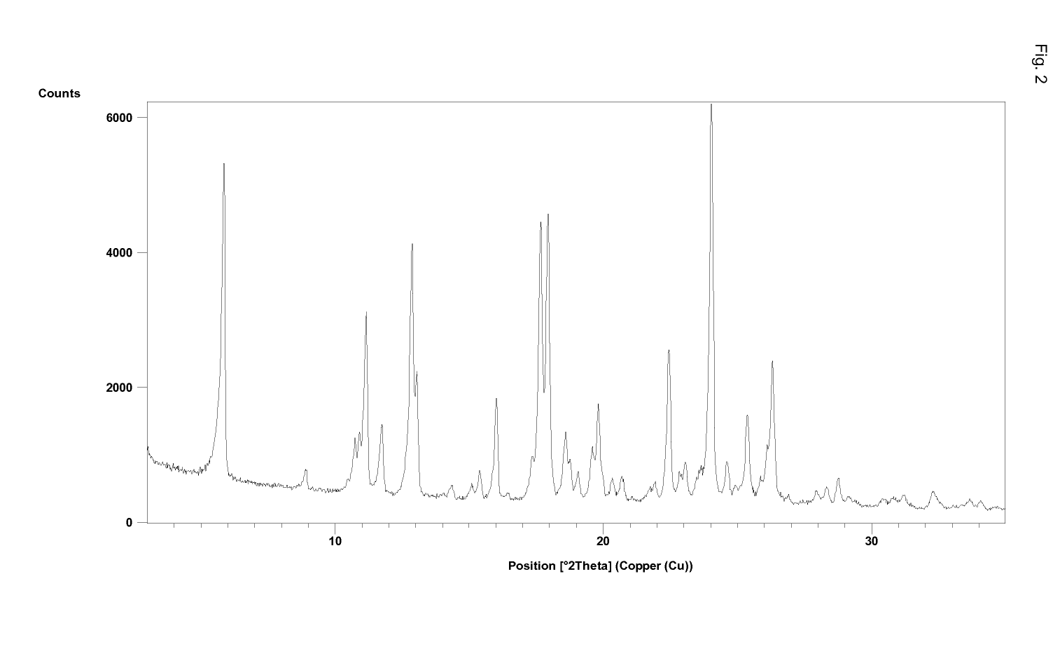

1. A process for obtaining a multicomponent crystals crystal comprising Dasatinib, which is a compound of formula 1 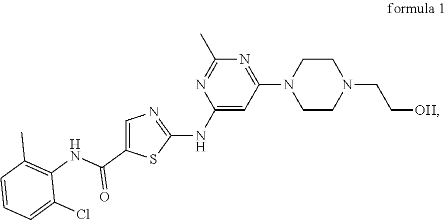 [SMILES: Cc1nc(Nc2ncc(C(=O)Nc3c(C)cccc3Cl)s2)cc(N2CCN(CCO)CC2)n1]
and
a second compound selected from the group consisting of menthol and vanillin,
the process comprising:
(a) providing the compound of formula 1; and
(b) adding at least a stoichiometric amount of menthol or vanillin to the compound of formula 1 such that the
menthol or vanillin is used as a co-crystal former and as a solvent to form a mixture that can be stirred,
or adding at least a stoichiometric amount of menthol or vanillin as a co-crystal former and an additional solvent to the compound of formula 1 such that a mixture is formed, wherein the additional solvent is selected from the group consisting of a C2 to C5 alcohol, a C3 to C6 ketone, a C2 to C6 ester, an ether, an alkane, and a mixture thereof.

2. A process according to claim 1, wherein the adding in (b) further comprises heating the mixture to a temperature of at least a melting temperature of the co-crystal former.

3. A process according to claim 1, wherein the multicomponent crystal is formed in a suspension or a concentrated solution of the menthol or vanillin in the additional solvent, or in a suspension or a concentrated solution of menthol in the absence of the additional solvent.

4. A process according to claim 1, further comprising:
(c) heating the mixture obtained in (b) to a temperature of from 40 to 150° C.;
(d) optionally cooling the mixture heated in (c) to a temperature from −10° C. to less than 30° C.;
(e) isolating a crystalline material; and
optionally washing the crystalline material using the menthol or vanillin or the additional solvent, drying the crystalline material, or both.

5. A process according to claim 1, wherein an amount of the menthol or vanillin added in (b) is at least 2 molar parts per 1 molar part of the compound of formula 1.

6. A process according to claim 1, wherein any solvent used in addition to the menthol or vanillin is selected from the group consisting of ethanol, propanol, acetone, methyl ethyl ketone, methyl isobutyl ketone, methyl acetate, ethyl acetate, propyl acetate, butyl acetate, ethyl formate, ethyl ether, isopropyl ether, methyl tert-butyl ether, pentane and heptane, and a mixture thereof.

7. A process according to claim 1, wherein the menthol is selected from the group consisting of (1R,2S,5R)-(−)-menthol, (1S,2R,5S)-(+)-menthol, DL-menthol, a stereoisomer of menthol, and a mixture thereof.

8. A multicomponent crystal, comprising:
a compound of formula 1 (INN: Dasatinib) 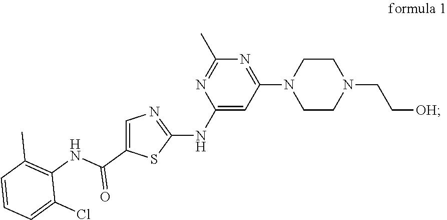 [SMILES: Cc1nc(Nc2ncc(C(=O)Nc3c(C)cccc3Cl)s2)cc(N2CCN(CCO)CC2)n1]
and
a second compound selected from the group consisting of menthol and vanillin,
wherein the multicomponent crystal is obtained or obtainable by the process according to claim 1.

9. A multicomponent crystal according to claim 8, wherein a molar ratio of the compound of formula 1 to the second compound is in a range of from 1.1:1 to 0.9:1 if the second compound is vanillin, and a molar ratio of the compound of formula 1 to the second compound is in a range of from 2.2:1 to 1.8:1 if the second compound is menthol.

10. A multicomponent crystal according to claim 8, wherein the second compound is menthol and the multicomponent crystal has a PXRD pattern comprising characteristic peaks, expressed in °2θ±0.2° 2θ, CuKα radiation, located at 5.8, 9.1, 10.4, 11.7, 11.9. 12.7, 14.9, 15.7 16.5. 18.2, 21.1, 21.3, 21.8, 22.8, 23.9, and 24.4.

11. A multicomponent crystal according to claim 8, wherein the second compound is vanillin and the multicomponent crystal has a PXRD pattern comprising characteristic peaks, expressed in °2θ±0.2° 2θ, CuKα radiation, located at 5.9, 8.9, 11.2, 11.8, 12.9, 15.4, 16.0, 17.7, 17.9, 18.6, 19.0, 19.8 20.7, 22.4, 24.0, 24.6, 25.4, and 26.3.

12. A composition, essentially consisting of:
the multicomponent crystal according to claim 8;
menthol or vanillin; and
up to 0.5% by weight, relative to a total weight of the composition, of the additional solvent.

13. A pharmaceutical composition, comprising:
an active ingredient comprising the multicomponent crystal according to claim 8; and
a pharmaceutically acceptable carrier, a diluent, a pharmaceutical excipient, or a mixture thereof.

14. A pharmaceutical composition according to claim 13, wherein a total amount of the multicomponent crystal per dosage unit is in a range of from 0.1 to 300 mg.

15. A method for treating cancer, comprising:
administering to a subject the multicomponent crystal according to claim 8, wherein the cancer is at least one of chronic myelogenous leukemia (CML) and Philadelphia chromosome-positive acute lymphoblastic leukemia (Ph+ ALL).

16. A method according to claim 1, wherein an amount of the menthol or vanillin added to the compound of formula 1 is substantially greater than an amount of menthol or vanillin in the multicomponent crystal.

17. The method according to claim 1, wherein the additional solvent comprises an ICH class 3 solvent.

18. A method according to claim 1, wherein the additional solvent is added in (b) such that suspension equilibration and filtration are carried out at a temperature lower than a melting temperature of the co-crystal former.

19. A process according to claim 1, wherein an amount of the menthol or vanillin added in (b) is about 1 to 20 parts by weight per one part by weight of the compound of formula 1.

20. A composition according to claim 12, wherein the composition contains less than 0.5% by weight of a component other than the multicomponent crystal, the menthol or vanillin, and the additional solvent.
